(19) United States
(12) Patent Application Publication
(10) Pub. No.: US 2008/0015572 A1
(43) Pub. Date: Jan. 17, 2008

(54) Method for energy-based stimulation of acupuncture meridians

(75) Inventors: Kristin D. Johnson, Louisville, CO (US); Ronald J. Podhajsky, Boulder, CO (US)

Correspondence Address: COVIDIEN, 60 MIDDLETOWN AVENUE, NORTH HAVEN, CT, 06473, US

(73) Assignee: SHERWOOD SERVICES AG

(21) Appl. No.: 11/487,296

(22) Filed: Jul. 14, 2006

(51) Int. Cl. A61B 18/18

(52) U.S. Cl. 606/44

(57) ABSTRACT

According to an aspect of the present disclosure, a method of weight loss involving acupuncture includes the steps of placing a plurality of EBS acupuncture needles at a plurality of predetermined locations of the body corresponding to acupuncture meridians; and energizing each of the plurality of EBS acupuncture needles to treat the body at the location of the EBS needle insertion.

### BACKGROUND

1. Technical Field

The present disclosure relates to surgical methods and, more particularly, to electrosurgical methods for energy-based stimulation of acupuncture meridians for weight loss and the like.

2. Background of Related Art

In the United States, excess weight or obesity is a problem for a majority of the population. A 1999 National Health and Nutrition Examination Survey concluded that 61% of American adults are overweight or obese and the percentage of people becoming overweight continues to rise. People who are overweight or obese are generally more prone to ailments such as high blood pressure, heart disease, and diabetes.

In order to lose weight and combat the conditions associated with excess weight, many individuals partake in numerous methods and/or procedures to lose weight. For example, individuals attempt diets, undertake exercise routines or regimens, purchase and use weight reduction equipment or weight reduction drugs, and the like.

Acupuncture has long been used to treat anxiety, back pain, high blood pressure, and osteoarthritis. People are now turning to acupuncture as a treatment for weight loss. Acupuncture is the ancient Chinese art of inserting fine needles under the surface of the skin into specific locations on the body to treat ailments and the like.

Acupuncturists have charted points along numerous paths, called meridians, on the body. Many of the meridians are considered to affect various body organs and body functions and, in turn, are important in treating various ailments. Typically, thin, solid needles are placed in one or several locations on the body called acupuncture points. The number of needles and the specific location of needle placement depends on the condition to be treated and on the severity of the condition. Acupuncturists treat these conditions by percutaneously inserting or placing needles into the various points associated with that condition.

### SUMMARY

The present disclosure relates to electrosurgical methods for energy-based stimulation of acupuncture meridians for weight loss and the like.

According to an aspect of the present disclosure, a method for performing a treatment on a human body includes the steps of placing a plurality of energy-based stimulation needles at a plurality of predetermined acupuncture meridian locations of the body; and energizing each of the plurality of energy-based stimulation needles to treat the body.

According to another aspect of the present disclosure, a method of weight loss involving acupuncture includes the steps of placing a plurality of energy-based stimulation acupuncture needles at a plurality of predetermined locations of the body corresponding to acupuncture meridians; and energizing each of the plurality of energy-based stimulation acupuncture needles to treat the body at the location of the energy-based stimulation needle insertion.

Either of the methods may include the step of connecting each energy-based stimulation needle to an energy-based generator. Either of the methods may further include the step of monitoring a temperature of the body at each location of energy-based stimulation needle placement.

Each of the methods may further include the step of energizing each energy-based stimulation needle with an RF waveform. The RF waveform may have a frequency above about 50 kHz. Each energy-based stimulation needle may be energized with one of a continuous wave and a pulsed wave.

Each of the methods may further include the step of maintaining a temperature of the body at each location of energy-based stimulation needle placement below a threshold level.

Either of the methods may further include the step of maintaining a temperature of the body at each location of energy-based stimulation needle placement below 42° C.

In either method, the energy-based stimulation needles may be placed at “stomach 31” (ST31), “stomach 34” (ST34), “stomach 43 (ST43), and/or “gall bladder 41” (GB41).

In either method, the energy-based stimulation needles may be placed at “spleen 9” (SP9), “spleen 8” (SP8) 16 and/or “spleen 6” (SP6).

Either method may further include placing the energy-based stimulation needles at “conception vessel 23” (CV23), “large intestine 11” (LI11), “conception vessel 12” (CV12), “stomach 36” (ST36), “spleen 6” (SP6), “spleen 10” (SP10) and “large intestine 3” (LI3), and/or “stomach 40” (ST40), “kidney 3” (KD3), “kidney 10” (KD10), “lung 9” (LU9) and “spleen 9” (SP9).

### BRIEF DESCRIPTION OF THE DRAWINGS

Further features and advantages of the invention will become readily apparent from the following specification and from the drawings, in which:

FIG. 1 is a schematic illustration of an energy-based stimulation system for performing an energy-based stimulation procedure of the present disclosure;

FIG. 2 is a schematic front view of a human torso showing locations for needle placement, along a conception vessel meridian, according to an embodiment of the present disclosure;

FIG. 3 is a schematic front view of a human torso showing locations for needle placement, along a stomach meridian, according to another embodiment of the present disclosure;

FIG. 4 is a schematic front view of a human torso showing locations for needle placement, along a large intestine meridian, according to still another embodiment of the present disclosure;

FIG. 5 is a schematic illustration of a ventral side of a human right forearm and hand showing locations for needle placement, along a pericardium meridian, according to yet another method of the present disclosure;

FIG. 6 is a schematic illustration of a ventral side of a human right forearm and hand showing locations for needle placement, along a lung meridian, according to a method of the present disclosure;

FIG. 7 is a schematic front illustration of a human right leg showing locations for needle placement, along the stomach meridian, according to another method of the present disclosure;

FIG. 8 is a schematic outside illustration of a human right leg showing locations for needle placement, along a gall bladder meridian, according to still another method of the present disclosure;

FIG. 9 is a schematic inside illustration of a human right leg showing locations for needle placement, along a spleen meridian, according to still another method of the present disclosure; and

FIG. 10 is a further schematic inside illustration of a human right leg showing locations for needle placement, along a kidney meridian, according to a further method of the present disclosure.

### DETAILED DESCRIPTION

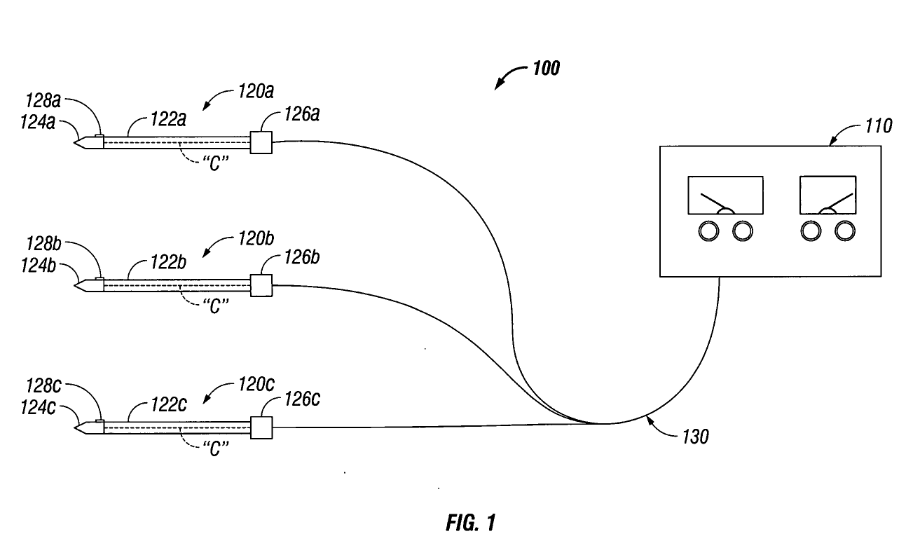

As seen in FIG. 1, an energy-based stimulation (EBS) system according to an embodiment of the present disclosure is generally designated as 100. EBS system 100 includes an EBS generator 110, at least one EBS acupuncture needle 120a-120c connectable to generator 110, and an electrical conduit 130 interconnecting each EBS needle 120a-120c to EBS generator 110.

EBS generator 110 is operable to generate and deliver any suitable therapeutic EBS energy to each EBS needle 120a-120c in order to deliver this energy to a target tissue, which may produce weight loss effects. In one embodiment, the therapeutic EBS energy may include a stimulating energy waveform or other suitable RF energy waveform having frequencies above approximately 50 kHz. In some embodiments, the stimulating energy waveform may include continuous or pulsed wave applications at any duty cycle. For example, the pulsed wave application may produce bursts of energy at a rate of approximately 2 bursts per second (i.e., 2 Hz of 20 msec duration each). Also, the tip of each EBS needle 120a-120c is to be maintained at a temperature level that is less than or equal to 42° C. for a period of 120 sec.

With continued reference to FIG. 1, each EBS needle 120a-120c includes a respective needle probe 122a-122c extending or extendable from a respective handle or hub 126a-126c. Each needle probe 122a-122c may be solid or hollow and may be fabricated from a suitable electrically conductive, rigid material, such as stainless steel. If needle probe 122a-122c is hollow, a cooling fluid may be circulated through a hollow cavity thereof in any suitable manner. An exterior surface of each needle probe 122a-122c may be coated with a suitable friction reducing material, such as TEFLON (PTFE) to aid in the percutaneous insertion of each needle probe 122a-122c into the body. Additionally, each needle probe 122a-122c may be coated with a suitable electrically insulative material along substantially an entire length thereof. A distal tip 124a-124c of each needle probe 122a-122c may be exposed for transmission of energy therefrom. Each distal tip 124a-124c may be sharpened in order to facilitate percutaneous tissue penetration and precise energy application, or may have an extended surface exposure to provide a broader area of effect.

As seen in FIG. 1, electrical conduit 130 may be connected to each needle probe 122a-122c via hubs 126a-126c. Alternatively, an end of electrical conduit 130 may be selectively electrically connected to at least one needle probe 122a-122c either prior to or after insertion of needle probe 122a-122c into the body. A clip (e.g., an alligator clip or other suitable connector) may be used to connect the end of electrical conduit 130 to a proximal end of needle probe 122a-122c.

Each EBS needle 120a-120c may include a respective temperature sensor 128a-128c disposed at a respective distal tip 124a-124c thereof. Each temperature sensor 128a-128c is electrically connected to EBS generator 110. Temperature sensors 128a-128c may form a component of a feedback control loop “C” capable of automatically adjusting an energy output of EBS generator 110. The energy output of EBS generator 110 may be adjusted so that the temperature of the body tissue in the proximity of distal tips 124a-124c of EBS needles 120a-120c does not exceed a threshold level that may result in local tissue damage, for example, 42° C.

In accordance with some embodiments of the present disclosure, EBS system 100 may be used to treat various ailments, including and not limited to weight management and/or weight loss. In operation, at least one EBS needle 120a-120c is placed at a specific treatment location on the human body, which specific treatment location coincides with known acupuncture meridians, and stimulated with energy.

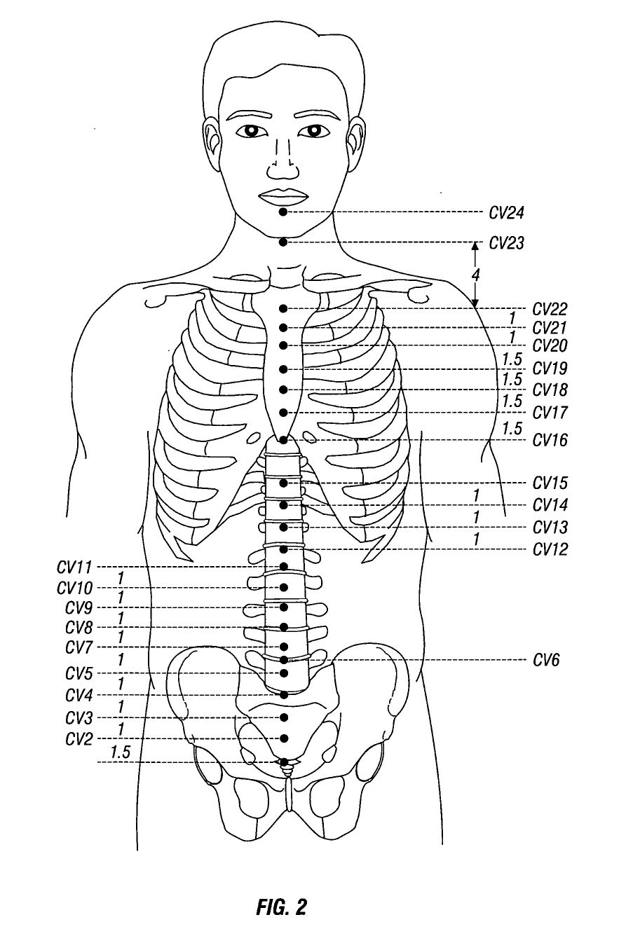

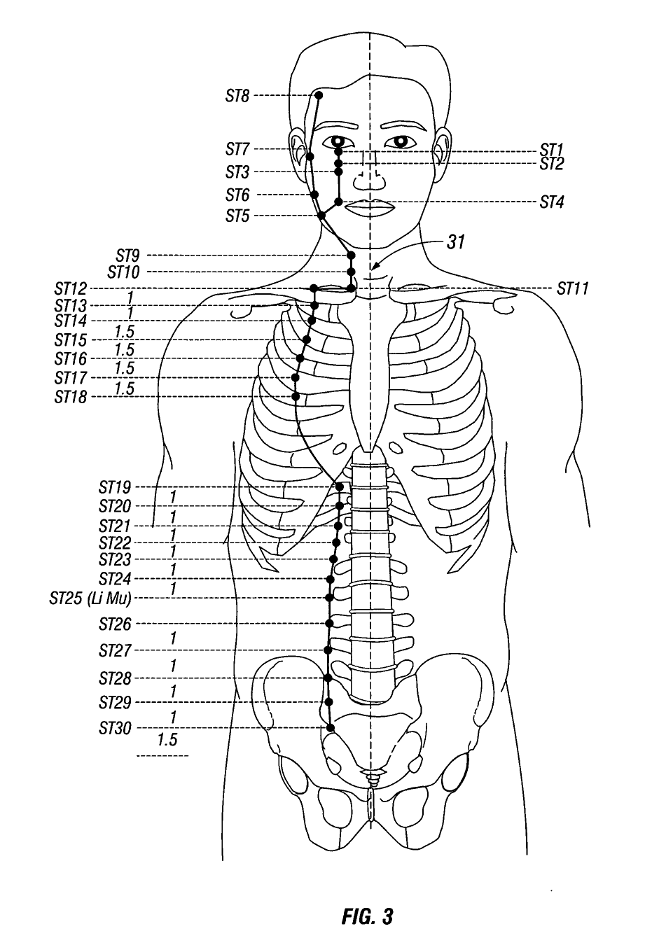

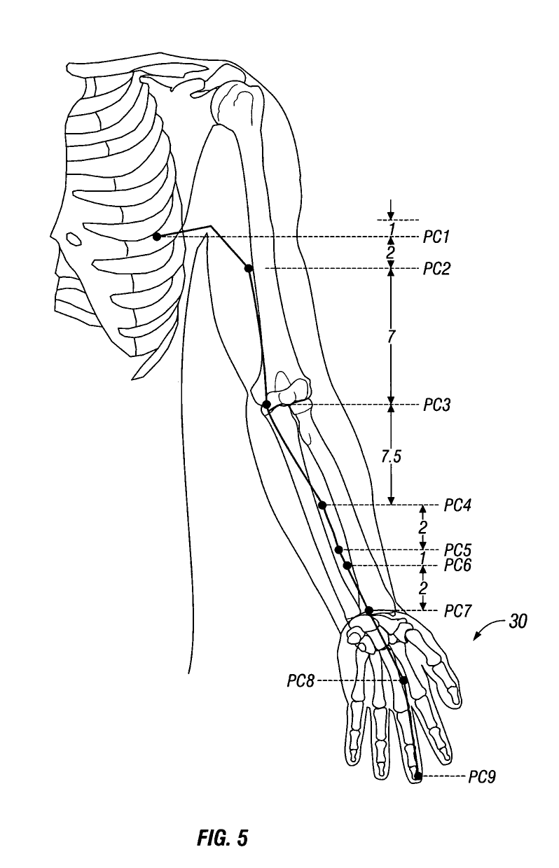

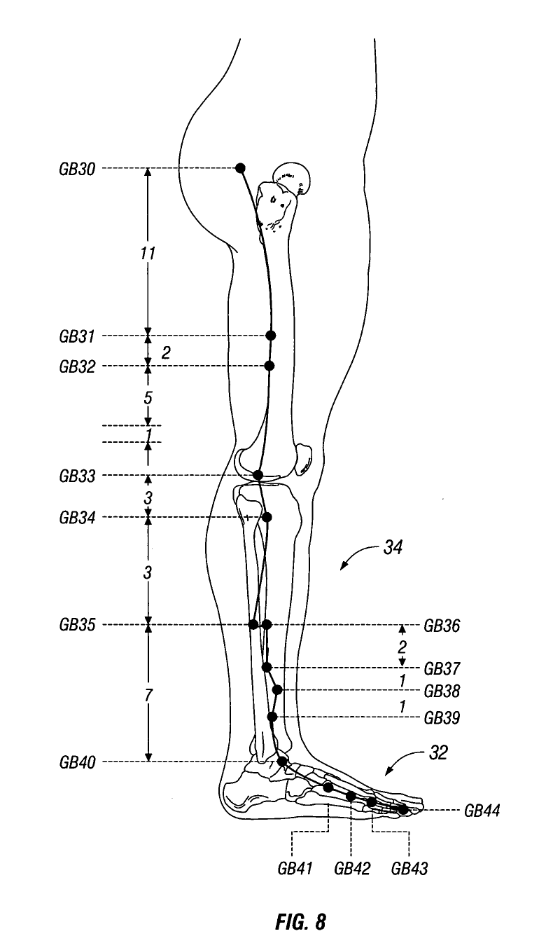

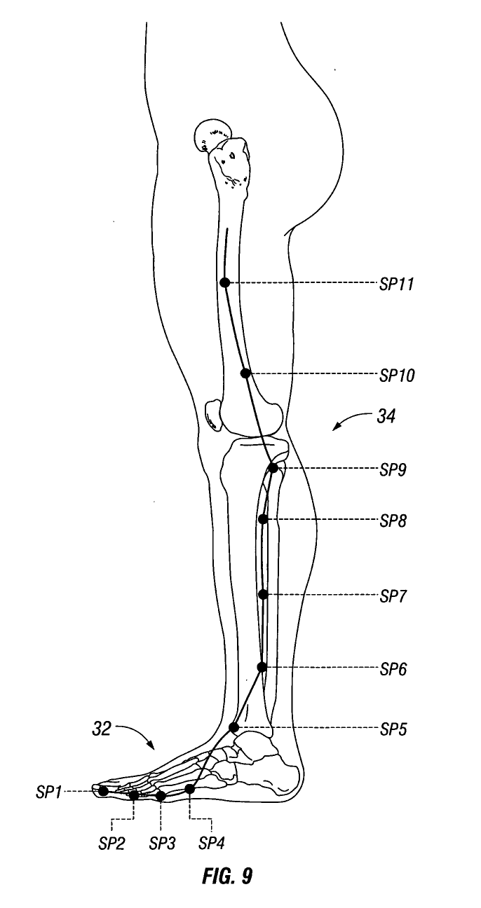

In one embodiment, as seen in FIGS. 2-10, treatment may include placement of at least one EBS needle 120a-120c on an anterior side (i.e., near a front) of a wrist 30, in the abdomen on one side of a medial line 31, and on an inside edge of the foot 32. A more precise location for the placement of EBS needles 120a-120c entails placing EBS needles 120a-120c at “pericardium 6” (PC6) of FIG. 5, “stomach 25” (ST25) of FIG. 3, and “spleen 4” (SP4) of FIG. 9.

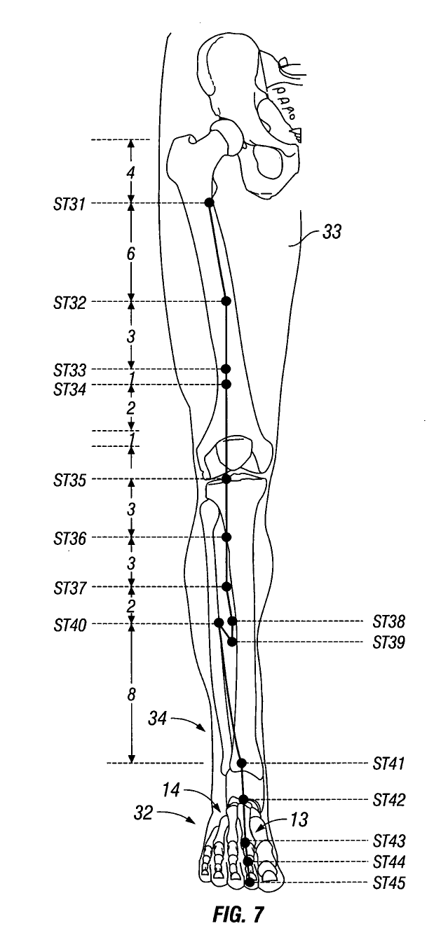

Depending on the circumstances and the condition of the patient, EBS needles 120 may be placed at several additional locations on the body for treatment of various ailments. For example, in another embodiment or according to a further method of the present disclosure, as seen in FIG. 7, EBS needles 120 may be placed at two additional locations on an anterior surface of the thigh 33, a first location, corresponding to “stomach 31” (ST31), located at the top of thigh 33 and a second location, corresponding to “stomach 34” (ST34), located in the thigh 33 just above the knee.

It is further envisioned, in certain situations, as seen in FIG. 7, for EBS needles 120 to be placed on an upper surface of the foot 32. For example, EBS needles 120 may be placed on a right-half side 14 of the upper surface of the foot 32 and/or on a left-hand side 13 of the upper surface of the foot 32.

In order to treat a patient for weight loss, EBS needles 120 may be placed at other locations in or on the body including, and not limited to, “stomach 31” (ST31), “stomach 34” (ST34), “stomach 43 (ST43), and “gall bladder 41” (GB41) of FIG. 7.

Additionally, in accordance with a further method of the present disclosure, as seen in FIG. 9, EBS needles 120 may be placed in or on an inside surface of a leg 34, a first EBS needle near the knee, a second EBS needle near mid-calf and/or a third EBS needle near the ankle. In particular, EBS needles 120 may be placed at “spleen 9” (SP9), at “spleen 8” (SP8) and/or at “spleen 6” (SP6).

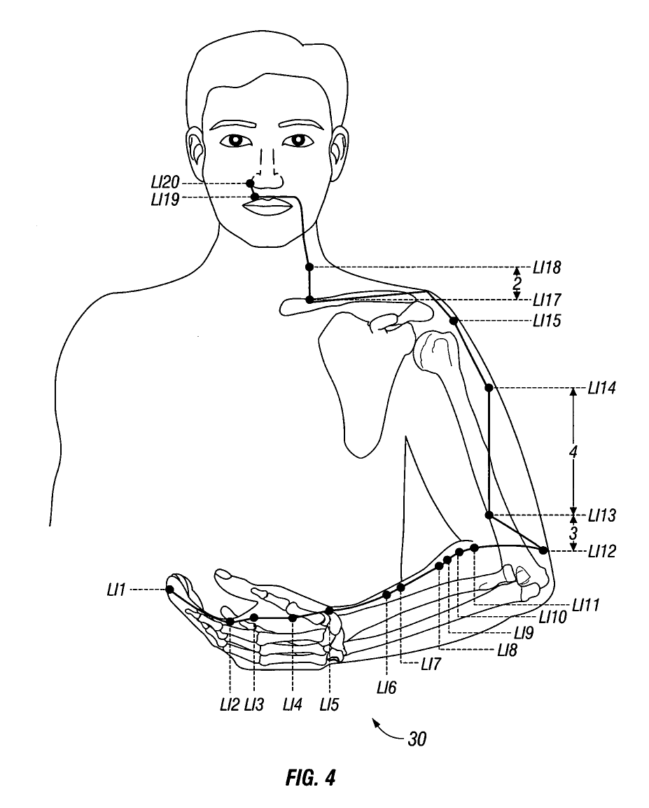

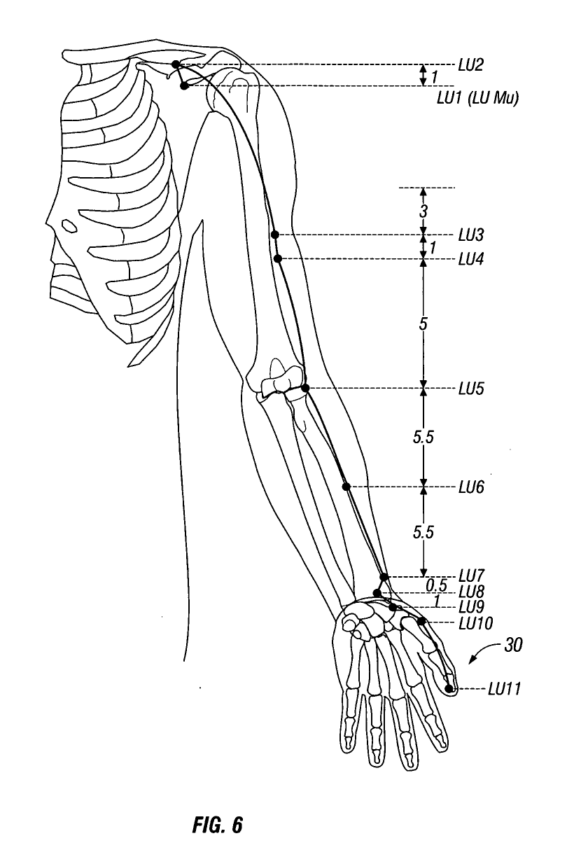

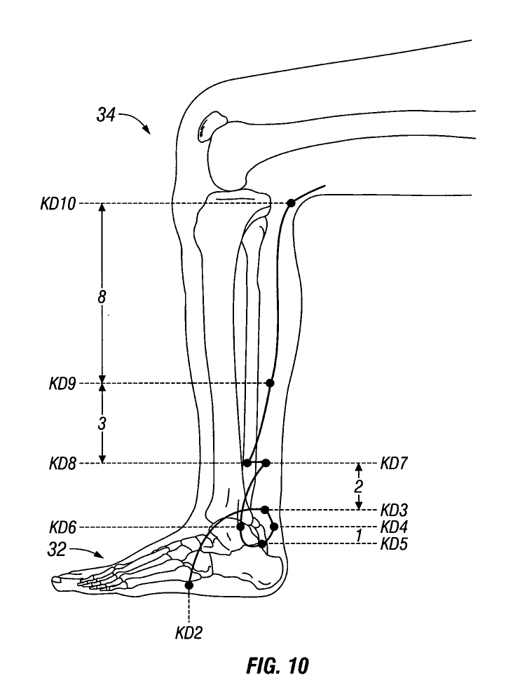

Other specific meridian locations that may be used for the insertion of an EBS needle 120, for the reduction of weight, include and are not limited to, “conception vessel 23” (CV23) for thyroid function as seen in FIG. 2; “large intestine 11” (LI11) for homeostasis as seen in FIG. 4; “conception vessel 12” (CV12) for stomach hyperactivity as seen in FIG. 2; “stomach 36” (ST36) for homeostasis as seen in FIG. 7; “spleen 6” (SP6) for hormone and fluid retention as seen in FIG. 9; “spleen 10” (SP10) as seen in FIG. 9 and “large intestine 3” (LI3) as seen in FIG. 4 for smooth blood flow regulation; and/or “stomach 40” (ST40) as seen in FIG. 7, “kidney 3” (KD3) as seen in FIG. 10, “kidney 10” (KD10) as seen in FIG. 10, “lung 9” (LU9) as seen in FIG. 6 and “spleen 9” (SP9) as seen in FIG. 9 for fluid removal.

The locations in the back, for example, “3 cun” (e.g., “1 cun” equals approximately 1.3 inches or 3.3 cm) lateral to a midline of the back are very beneficial for psycho-emotional, obsessive compulsive disorders and anorexia. It is further understood that “3 cun” may equal the measurement across four fingers of a hand, from pinky finger to index finger, when the fingers are held together.

While FIGS. 2-10 illustrate locations for placement of EBS needles 120 on a right-hand side of the body, those skilled in the art realize that these needle placement locations are also mirrored on a left-hand side of the body. Accordingly, when a location for placement of an EBS needle 120 is disclosed, said location may be applied to either the right-hand side of the body or the left-hand side of the body.

Following placement of EBS needles 120 electrosurgical energy is transmitted from EBS generator 110 to each EBS needle 120. For example, each EBS needle 120 may be stimulated with electrical or electrosurgical current and/or waveforms including frequencies above about 50kHz and may include either continuous wave or pulsed wave application at any duty cycle.

During application of EBS energy to EBS needles 120, sensors 128 may monitor the temperature at each insertion site and provide feedback, via control loop “C”, to EBS generator 110. EBS generator 110 may be configured to adjust the parameters of the EBS energy transmitted to EBS needles 120 in response to the feedback from sensors 128. EBS generator 110 may adjust the parameters of the EBS energy being transmitted so that the temperature of the tissue does not exceed a threshold which may result in local tissue damage. The threshold temperature for the tissue may not exceed about 42° C.

The foregoing description is merely a disclosure of particular embodiments and is no way intended to limit the scope of the invention. Other possible modifications are apparent to those skilled in the art and all modifications are to be defined by the following claims.

What is claimed is:

1. A method for performing a treatment on a human body, the method comprising the steps of:
placing a plurality of EBS needles at a plurality of respective predetermined acupuncture meridian locations of the body; and
energizing each of the plurality of EBS needles to treat the body.

2. The method according to claim 1, further comprising the step of connecting each EBS needle to an EBS generator.

3. The method according to claim 1, further comprising the step of monitoring a temperature of the body at each location of EBS needle placement.

4. The method according to claim 1, wherein the energizing step comprises energizing each EBS needle with an RF waveform.

5. The method according to claim 1, further comprising the step of energizing each EBS needle with an RF waveform having a frequency above about 50 kHz.

6. The method according to claim 1, wherein the energizing step comprises energizing each EBS needle with one of a continuous wave and a pulsed wave in at least one of a simultaneous and serial manner.

7. The method according to claim 3, further comprising the step of maintaining a temperature of the body at each location of EBS needle placement below a threshold level.

8. The method according to claim 3, further comprising the step of maintaining a temperature of the body at each location of EBS needle placement below 42° C.

9. The method according to claim 1, wherein the EBS needles are placed at at least one of “stomach 31” (ST31), “stomach 34” (ST34), “stomach 43 (ST43), and “gall bladder 41” (GB41).

10. The method according to claim 9, wherein the EBS needles are placed at at least one of “spleen 9” (SP9), “spleen 8” (SP8) 16 and “spleen 6” (SP6).

11. The method according to claim 10, wherein the EBS needles are placed at at least one of “conception vessel 23” (CV23), “large intestine 11” (LI11), “conception vessel 12” (CV12), “stomach 36” (ST36), “spleen 6” (SP6), “spleen 10” (SP10) and “large intestine 3” (LI3), and “stomach 40” (ST40), “kidney 3” (KD3), “kidney 10” (KD10), “lung 9” (LU9) and “spleen 9” (SP9).

12. A method of weight loss involving acupuncture, comprising the steps of:
placing a plurality of EBS acupuncture needles at a plurality of respective predetermined locations of the body corresponding to acupuncture meridians; and
energizing each of the plurality of EBS acupuncture needles to treat the body at the location of the EBS needle insertion.

13. The method according to claim 12, further comprising the step of connecting each EBS needle to an EBS generator.

14. The method according to claim 12, further comprising the step of monitoring a temperature of the body at each location of EBS needle placement.

15. The method according to claim 12, further comprising the step of energizing each EBS needle with an RF waveform.

16. The method according to claim 12, further comprising the step of energizing each EBS needle with an RF waveform having a frequency above about 50kHz.

17. The method according to claim 12, further comprising the step of energizing each EBS needle with one of a continuous wave and a pulsed wave.

18. The method according to claim 12, further comprising the step of maintaining a temperature of the body at each location of EBS needle placement below a threshold level.

19. The method according to claim 12, further comprising the step of maintaining a temperature of the body at each location of EBS needle placement below 42° C.

20. The method according to claim 12, wherein the EBS needles are placed at at least one of “stomach 31” (ST31), “stomach 34” (ST34), “stomach 43 (ST43), and “gall bladder 41” (GB41).

21. The method according to claim 12, wherein the EBS needles are placed at at least one of “spleen 9” (SP9), “spleen 8” (SP8) 16 and “spleen 6” (SP6).

22. The method according to claim 12, wherein the EBS needles are placed at at least one of “conception vessel 23” (CV23), “large intestine 11” (LI11), “conception vessel 12” (CV12), “stomach 36” (ST36), “spleen 6” (SP6), “spleen 10” (SP10) and “large intestine 3” (LI3), and “stomach 40” (ST40), “kidney 3” (KD3), “kidney 10” (KD10), “lung 9” (LU9) and “spleen 9” (SP9).
